(19) United States
(12) Patent Application Publication
(10) Pub. No.: US 2026/0114875 A1
(43) Pub. Date: Apr. 30, 2026

(54) LEFT ATRIAL APPENDAGE LIGATION DEVICE

(71) Applicant: BOSTON SCIENTIFIC SCIMED, INC., MAPLE GROVE, MN (US)

(72) Inventors: Jerry Wang, Edina, MN (US); Joshua Mark Inouye, Brooklyn Park, MN (US); James M. Anderson, Corcoran, MN (US)

(73) Assignee: BOSTON SCIENTIFIC SCIMED, INC., MAPLE GROVE, MN (US)

(21) Appl. No.: 19/421,665

(22) Filed: Dec. 16, 2025

Related U.S. Application Data
(63) Continuation of application No. 18/747,730, filed on Jun. 19, 2024.
(60) Provisional application No. 63/524,285, filed on Jun. 30, 2023.

(51) Int. Cl. A61B 17/12; A61B 17/00

(52) U.S. Cl. CPC A61B 17/12122; A61B 17/12036; A61B 17/12136; A61B 17/1214; A61B 2017/00632; A61B 2017/00867; A61B 2017/12054

(57) ABSTRACT

A left atrial appendage (LAA) ligation device is adapted for ligating the LAA via a trans-septal approach, the LAA including an ostium. The LAA ligation device includes a spiral ligation element extending from an end point disposed at a periphery of the spiral ligation element to a center point of the spiral ligation element, the spiral ligation element having a diameter defined by the periphery and a length defined between the periphery and the center point. A delivery device is adapted to releasably secure the spiral ligation element and to enable rotation of the spiral ligation device.

### CROSS REFERENCE TO RELATED APPLICATIONS

This application is a continuation of U.S. application Ser. No. 18/747,730, filed Jun. 19, 2024, which claims the benefit of priority of U.S. Provisional Application No. 63/524,285 filed Jun. 30, 2023, the entire disclosure of which is hereby incorporated by reference.

### TECHNICAL FIELD

The disclosure relates generally to medical devices and more particularly to medical devices that are adapted for detecting leaks around a left atrial appendage closure device.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a left atrial appendage (LAA) ligation device adapted for ligating the LAA via a trans-septal approach, the LAA including an ostium. The LAA ligation device includes a spiral ligation element extending from an end point disposed at a periphery of the spiral ligation element to a center point of the spiral ligation element, the spiral ligation element having a diameter defined by the periphery and a length defined between the periphery and the center point. A delivery device is adapted to releasably secure the spiral ligation element and to enable rotation of the spiral ligation device.

Alternatively or additionally, the end point of the spiral ligation element may be adapted to penetrate through the ostium of the LAA.

Alternatively or additionally, rotation of the spiral ligation device after the end point of the spiral ligation element has penetrated through the ostium of the LAA may cause the spiral ligation device to wrap around an exterior of the LAA, thereby cinching down onto the LAA.

Alternatively or additionally, the delivery device may be adapted to releasably secure the center point of the spiral ligation element.

Alternatively or additionally, the center point of the spiral ligation element may include a first releasable locking feature and the delivery device may include a second releasable locking feature complementary to the first releasable locking feature.

Alternatively or additionally, the LAA ligation device may further include a removable sheath that holds the first releasable locking feature engaged with the second releasable locking feature until the removable sheath is removed.

Alternatively or additionally, the spiral ligation element may be held to the delivery device via a weakened spot that is adapted to break upon application of sufficient torque.

Alternatively or additionally, the delivery device may include a dual-wound coil or a slotted laser-cut hypotube.

Alternatively or additionally, the LAA ligation device may further include a polymeric sealing member disposed relative to the spiral ligation element.

Alternatively or additionally, the spiral ligation element may be formed from an elongate member having a circular cross-sectional profile.

Alternatively or additionally, the spiral ligation element may be formed from an elongate member having a rectilinear cross-sectional profile.

Another example may be found in a left atrial appendage (LAA) ligation device adapted for ligating the LAA via a trans-septal approach, the LAA including an ostium. The LAA ligation device includes a spiral ligation element extending from an endpoint disposed at a periphery of the spiral ligation element to a center point of the spiral ligation element, the spiral ligation element having a diameter defined by the periphery and a length defined between the periphery and the center point, the spiral ligation element defining a lumen extending through the spiral ligation element. A penetration element is slidingly disposed within the lumen, the penetration element adapted for penetrating through an ostium of the LAA. A delivery device is adapted to releasably secure the spiral ligation element and to enable rotation of the spiral ligation device.

Alternatively or additionally, the endpoint may be adapted to curl into an atraumatic tip when the penetration element is withdrawn from the lumen.

Alternatively or additionally, the LAA ligation device further includes an atraumatic element adapted to be advanced through the lumen and extend out of the lumen at the endpoint subsequent to removal of the penetration element.

Alternatively or additionally, the atraumatic element may include an elongate member having a distal end and an atraumatic pigtail at the distal end.

Alternatively or additionally, the atraumatic element may include an elongate member having a distal end and a balloon disposed at the distal end.

Alternatively or additionally, the atraumatic element may include an elongate member having a distal end and an expandable nitinol member disposed at the distal end.

Alternatively or additionally, the spiral ligation element may be biased to a first configuration in which the spiral ligation element has a first overall diameter and the spiral ligation element may be adapted to be expanded to a second configuration having a second overall diameter greater than the first overall diameter by extending a stiff wire through the lumen.

Another example may be found in a left atrial appendage (LAA) ligation device adapted for ligating the LAA via a trans-septal approach, the LAA including an ostium. The LAA ligation device includes a first spiral element defining a lumen extending therethrough and a second spiral element adapted to be advanced through the lumen of the first spiral element and extend out a distal end of the lumen in order to create a larger spiral. The second spiral element is adapted to penetrate the ostium of the LAA and to wind around an exterior of the LAA as the LAA ligation device is rotated. The first spiral element is adapted to cinch down onto the LAA as the LAA ligation device is further rotated, and to secure the LAA once the second spiral element is removed from the first spiral element.

Alternatively or additionally, the first spiral element may be biased to a first configuration, and the second spiral element may be adapted to cause the first spiral element to expand to a second configuration when the second spiral element extends through the first spiral element.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional view of an LAA (left atrial appendage);

FIG. 2 is a perspective view of an illustrative LAA ligation device;

FIGS. 2A through 2C are representative cross-sections taken along the line 2A-2A of FIG. 2, each showing a different possible profile for the elongate member forming the LAA ligation device;

FIGS. 3A through 3D provide an example of how an LAA ligation device constricts the LAA;

FIG. 4 is a side view of an illustrative LAA ligation device;

FIG. 5A is a schematic view of an illustrative LAA ligation device;

FIG. 5B is a schematic view showing a releasable connection between the illustrative LAA ligation device of FIG. 5A and a delivery device;

FIG. 5C is a cross-sectional view taken along the line 5C-5C of FIG. 5B;

FIG. 6A is a schematic view of an illustrative LAA ligation device in a sheathed configuration;

FIG. 6B is a schematic view of the illustrative LAA ligation device of FIG. 6A in an unsheathed configuration;

FIG. 7 is a schematic view of an illustrative LAA ligation device;

FIGS. 8A through 8C are schematic views of illustrative shafts forming part of an illustrative LAA ligation device;

FIGS. 9A through 9D are schematic views of an illustrative LAA ligation device;

FIGS. 10A through 10D are schematic views of an illustrative LAA ligation device;

FIGS. 11A through 11E are schematic views of illustrative tips used with an illustrative LAA ligation device; and

FIG. 12 is a schematic view of an illustrative pericardium drainage procedure.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following figures illustrate selected components and/or arrangements of an implant for occluding the left atrial appendage, a system for occluding the left atrial appendage, and/or methods of using the implant and/or the system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

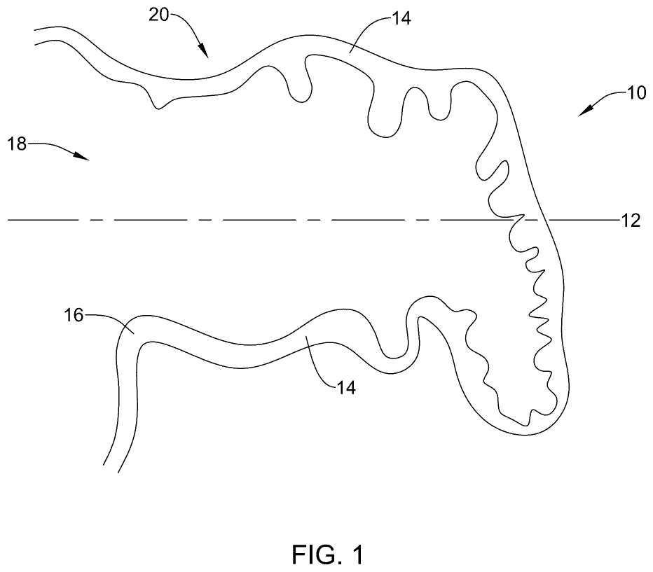

FIG. 1 is a partial cross-sectional view of a left atrial appendage 10. In some embodiments, the left atrial appendage (LAA) 10 may have a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12.

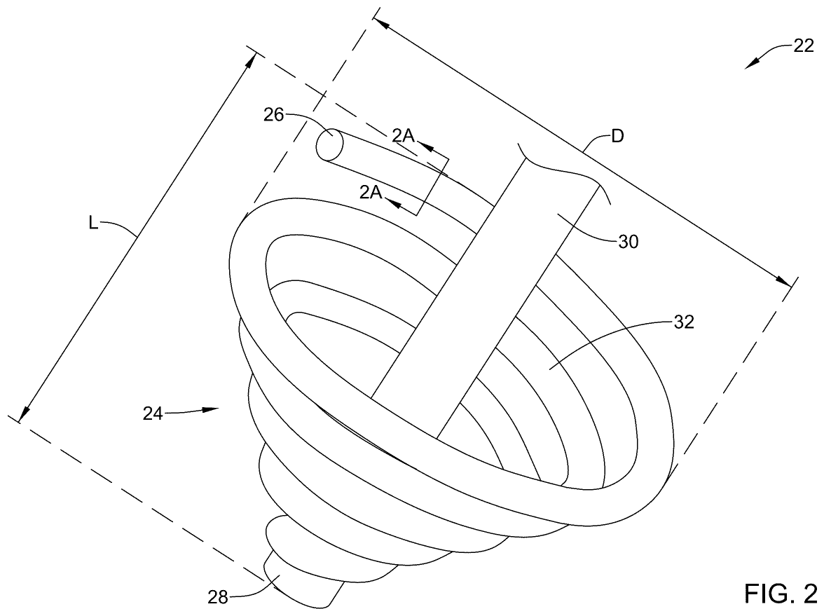

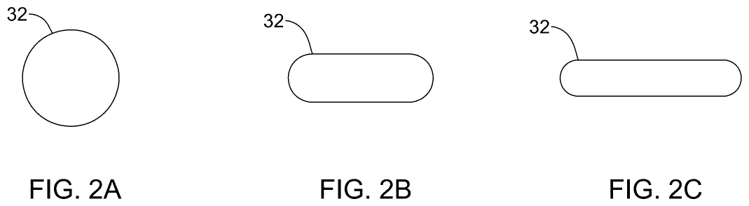

FIG. 2 is a perspective view of an illustrative LAA (left atrial appendage) ligation device 22 that may be used in closing off the LAA 10. In some instances, the LAA ligation device 22 may be used to close off the LAA 10 via a trans-septal approach in which the LAA ligation device 22 may be delivered to the LAA 10 from the right atrium, passing through the atrial septum, and into the left atrium. It will be appreciated that this may be considered as being an endocardial approach, which may be considered less invasive than an epicardial approach. In some instances, the LAA ligation device 22 may be used to pinch off or otherwise close the LAA 10 from a position within the LAA 10. In some instances, the LAA ligation device 22 may pierce the LAA 10, such as the ostium 16, from a position within the LAA 10. The LAA ligation device 22 may be rotated relative to the ostium 16. As the LAA ligation device 22 rotates, an increasing portion of the LAA ligation device 22 will pass through an aperture formed in the ostium 16 to a position exterior to the LAA 10. As the spiral-shaped LAA ligation device 22 rotates relative to the LAA 10, the spiral-shaped LAA ligation device 22 has a decreasing diameter, meaning that as the spiral-shaped LAA ligation device 22 rotates, the LAA 10 is increasingly pinched or cinched off.

The LAA ligation device 22 includes a spiral ligation element 24 that extends from an end point 26 that is disposed at a periphery of the spiral ligation element 24 to a center point 28 of the spiral ligation element 24. In some instances, the spiral ligation element 24 may be considered to be a closed spiral, in which multiple windings of the spiral ligation element 24 are coplanar, or within a single plane. In some instances, the spiral ligation element 24 may be considered as being an open spiral, in which each successive winding is axially spaced from a previous coiling. In some instances, particularly when the spiral ligation element 24 is an open spiral, as shown, the spiral ligation element 24 may have a diameter D that is defined by the periphery and a length L that is defined as the distance between the periphery and the center point 28. In some instances, the diameter D may be selected to accommodate an LAA 10 having a diameter of 18 millimeters to 40 millimeters, for example. In some instances, the diameter D (measured in a free state) may range from 10 millimeters to 50 millimeters. In some instances, the length L (measured in a free state) may range from 0 millimeters (spiral ligation element 24 is within the same plane) to 30 millimeters.

In some instances, the spiral ligation element 24 may be releasably secured to a delivery device 30, schematically shown in FIG. 2. In some instances, the delivery device 30 may be adapted to enable rotation of the spiral ligation element 24 by rotating the delivery device 30. In some instances, the delivery device 30 may be adapted to break upon application of sufficient torque, in order to release the spiral ligation element 24 from the delivery device 30. In some instances, as will be discussed with respect to subsequent drawings, the LAA ligation device 22 may include a lock and key interaction between corresponding structures formed in or on the spiral ligation element 24, such as at or near the center point 28, and the delivery device 30. In some instances, a retractable sheath may hold the corresponding structures together until such time as release is desired, in which case the retractable sheath may be withdrawn in order to allow the corresponding structures to disengage from one another.

In some instances, the end point 26 of the spiral ligation element 24 is adapted to penetrate through the ostium 16 of the LAA 10. In some instances, the end point 26 may be sharpened, for example. In some instances, the spiral ligation element 24 may be electrically conductive, and may be adapted to conduct RF (radio frequency) energy through the end point 26 in order to use RF energy to form an aperture through the ostium 16 of the LAA 10. In some instances, a separate element (not shown), such as a needle or a sharp wire, may be used to form the aperture. Rotation of the spiral ligation device 24 after an aperture has been formed through the ostium 16 of the LAA 10 may cause the end point 26 of the spiral ligation element 24 to extend through the aperture and to start wrapping around an exterior of the LAA 10. It will be appreciated that each successive winding, when moving from the end point 26 of the spiral ligation element 24 towards the center point 28, decreases in diameter. As a result, as the spiral ligation element 24 continues to advance through the aperture and wraps around the exterior of the LAA 10, the LAA 10 will be increasingly pinched or cinched down. Sufficient rotation of the spiral ligation element 24 will cause the LAA 10 to become closed off. At this point, the spiral ligation element 24 may be separated from the delivery device 30, and the delivery device 30 may be removed from the patient while the spiral ligation element 24 remains in place within the patient, and may be positioned partially within the LAA 10 and partially outside of the LAA 10, depending on how many rotations of the spiral ligation element 24 were needed to close off the LAA 10.

The spiral ligation element 24 may be formed from an elongate member 32 that is formed into a spiral shape as shown. The elongate member 32 may have a diameter ranging from 0.2 millimeters to 2 millimeters. In some instances, as shown in FIG. 2A, the elongate member 32 may have a circular cross-sectional profile. In some instances, as shown in FIG. 2B, the elongate member 32 may have a rectilinear cross-sectional profile. In some instances, as shown in FIG. 2C, the elongate member 32 may have a flattened cross-sectional profile, such as a flat ribbon. Other cross-sectional profiles are also contemplated. In some instances, differing cross-sectional profiles may provide various performance benefits. As an example, a circular cross-sectional profile, as shown for example in FIG. 2A, may provide for more uniform bending characteristics in any direction. A flat ribbon profile, such as that shown in FIG. 2C, may provide spatial benefits when the spiral ligation element 24 is collapsed for delivery. The elongate member 32 may be formed of any suitable metallic or polymeric material. Examples of suitable materials include but are not limited to shape memory materials, shapeable materials, and combinations and composites thereof. Examples include nickel titanium alloys, stainless steel, and various polymers such as elastomers and fluoropolymers.

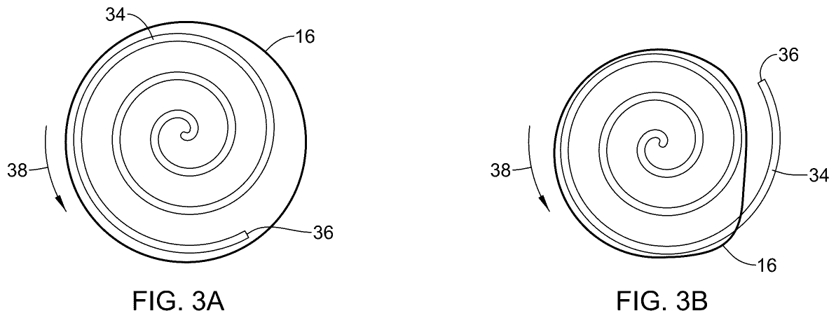

FIGS. 3A through 3D provide an illustrative example of how an LAA ligation device (such as the LAA ligation device 22) may be used to close off the LAA 10. FIG. 3A shows a spiral ligation element 34 disposed within an interior of the ostium 16 of the LAA 10. The spiral ligation element 34 includes an end point 36. The spiral ligation element 34 may be a closed spiral or an open spiral, for example. Moving to FIG. 3B, it can be seen that the spiral ligation element 34 has been rotated in a direction indicated by an arrow 38. Moving from FIG. 3A to 3B, it can be seen that the spiral ligation element 34 has rotated about 90 degrees or so, or about ¼ of a rotation, as can be seen by the relative location in each drawing of the end point 36. It can be seen in FIG. 3B that part of the spiral ligation element 34 is now exterior to the LAA 10, and the ostium 16 of the LAA 10 is starting to be compressed.

Moving from FIG. 3B to FIG. 3C, it can be seen that the spiral ligation element 34 has rotated about 90 degrees or so, or about ¼ of a rotation, as can be seen by the relative location in each drawing of the end point 36. It can be seen in FIG. 3C that an increasing part of the spiral ligation element 34 is now exterior to the LAA 10, and the ostium 16 of the LAA 10 is becoming further compressed. Moving from FIG. 3C to FIG. 3D, it can be seen that the spiral ligation element 34 has rotated about 90 degrees or so, or about ¼ of a rotation, as can be seen by the relative location in each drawing of the end point 36. It can be seen in FIG. 3D that an increasing part of the spiral ligation element 34 is now exterior to the LAA 10, and the ostium 16 of the LAA 10 is becoming further compressed. As rotation of the spiral ligation element 34 continues, it will be appreciated that the spiral ligation element 34 will continue to exit the ostium 16 of the LAA 10, and will continue to further constrict the LAA 10. While rotation in one direction (as indicated by the arrow 38) is shown, it will be appreciated that depending on which direction the spiral ligation element 34 is wound, the spiral ligation element 34 may be rotated in a direction opposite that shown by the arrow 38 in order to constrict the LAA 10.

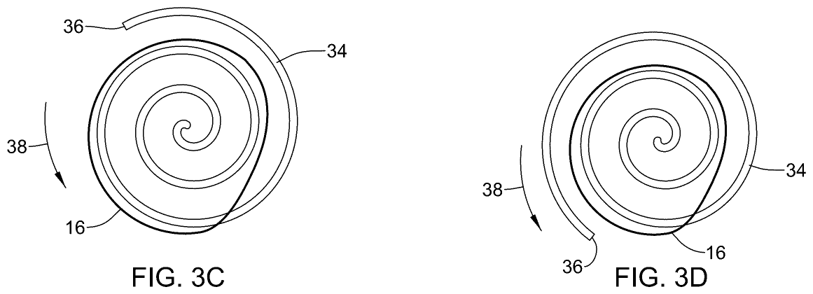

FIG. 4 is a side view of an illustrative LAA ligation device 40 that may be used to close off the LAA 10. The illustrative LAA ligation device 40 includes a spiral ligation element 42 has an end point 44. As shown, the spiral ligation element 42 may be considered as being a closed spiral, meaning that the spiral ligation element 42 largely lies within a single plane. In some instances, the spiral ligation element 42 may be an open spiral, having a greater length. In some instances, the spiral ligation element 42 may include an attachment member 46 that is secured relative to a center point 48 of the spiral ligation element 42. In some instances, the attachment member 46 provides a surface upon which the LAA 10 is compressed against when the spiral ligation element 42 is rotated relative to the LAA 10. In some instances, the attachment member 46 also provides a releasable connection to a delivery device 50.

As shown, the attachment member 46 includes a first locking feature 52 and the delivery device 50 includes a corresponding and complementary second locking feature 54 that is adapted to releasably engage with the first locking feature 52 in order to secure the spiral ligation element 42 relative to the delivery device 50. In some instances, the first locking feature 52 and the second locking feature 54 engage to provide a connection by which the spiral ligation element 42 may be translated and/or rotated by translating and/or rotating the delivery device 50 as long as the first locking feature 52 and the second locking feature 54 are held together. In some instances, the delivery device 50 may include a sheath 56 that may be moved between an advanced position in which the sheath 56 covers both the first locking feature 52 and the second locking feature 54, thereby holding the first locking feature 52 and the second locking feature 54 together, and a retracted position (as shown) in which the sheath 56 does not cover the first locking feature 52 or the second locking feature 54, and as such the first locking feature 52 may be free to move relative to the second locking feature 54. Accordingly, the spiral ligation element 42 may be released from the delivery device 50 simply by retracting the sheath 56.

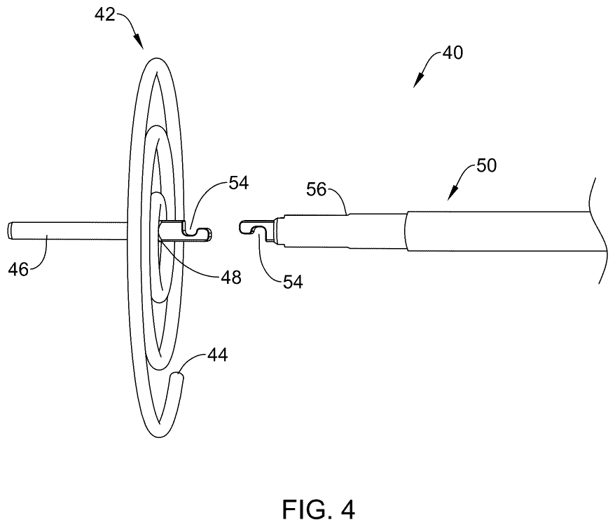

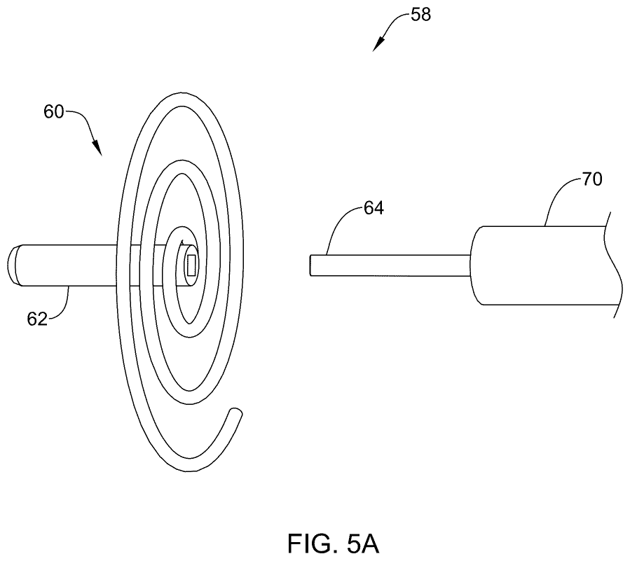

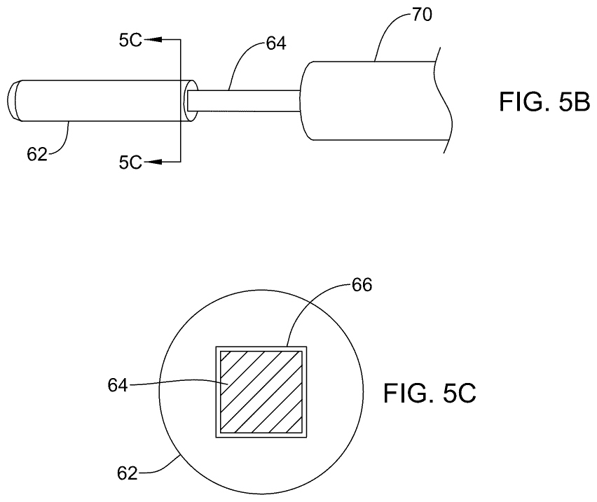

FIG. 5A is a schematic view of an illustrative LAA ligation device 58. The illustrative LAA ligation device 58 includes a spiral ligation element 60. The spiral ligation element 58 may be considered as either a closed spiral, meaning that the spiral ligation element 58 largely lies within a single plane, or open spiral, having a greater length. In some instances, the spiral ligation element 58 may include an attachment member 62 that is secured to the spiral ligation element 60. In some instances, the attachment member 62 provides a surface upon which the LAA 10 is compressed against when the spiral ligation element 60 is rotated relative to the LAA 10. In some instances, the attachment member 62 also provides a releasable connection to a delivery shaft 64, which forms part of a delivery device.

FIG. 5B is an enlarged view of the attachment member 62 and the delivery device 64. In some instances, the attachment member 62 includes an aperture 66 that is adapted to accommodate the delivery shaft 64. In some instances, there may be a complementary lock and key arrangement between the aperture 66 and the delivery shaft 64 such that rotation of the delivery shaft 64 results in rotation of the attachment member 62 (and hence rotation of the spiral ligation element 60. In some instances, the aperture 66 may have a non-circular shape that matches an outer profile of the delivery shaft 64. As a result, the delivery shaft 64 is not able to rotate within the aperture 66. In some instances, as shown, the aperture 66 may have a rectilinear profile, and the delivery shaft 64 or at least a distal end of the delivery shaft 64 may have a similar rectilinear profile that is complementary to that of the aperture 66.

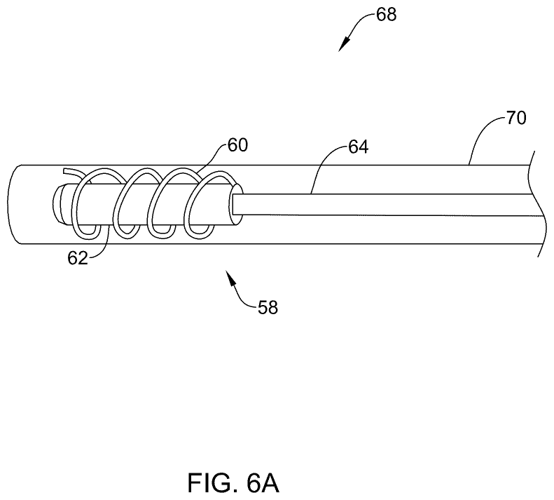

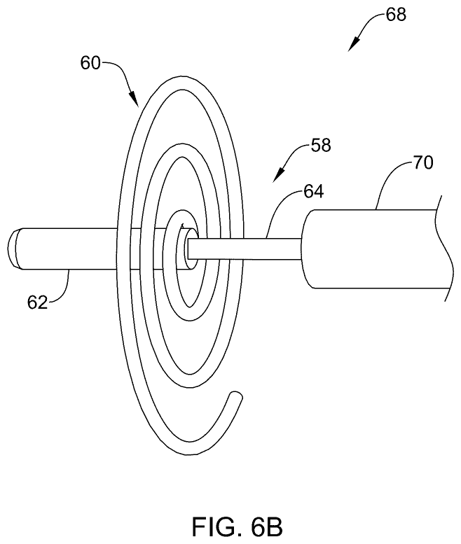

FIG. 6A is a schematic view of an assembly 68 that includes the illustrative LAA ligation device 58 disposed within a delivery sheath 70. FIG. 6A shows the assembly 68 with the LAA ligation device 58 sheathed within the delivery sheath 70. It can be seen that the spiral ligation element 60 wraps around the attachment member 62. FIG. 6B shows the assembly 68 with the LAA ligation device 58 advanced distally relative to the delivery sheath 70 (or alternatively, with the delivery sheath 70 retracted proximally relative to the LAA ligation device 58). It will be appreciated that the delivery shaft 64 and the delivery sheath 70 may both be parts of a delivery device.

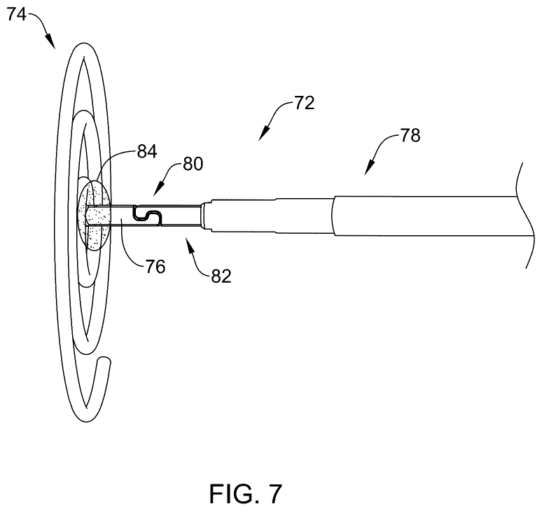

FIG. 7 is a schematic view of an illustrative LAA ligation device 72 that may be used to close off the LAA 10. The illustrative LAA ligation device 72 includes a spiral ligation device 74 that includes an attachment member 76. The spiral ligation element 74 may be considered as either a closed spiral, meaning that the spiral ligation element 74 largely lies within a single plane, or open spiral, having a greater length. The LAA ligation device 72 includes a delivery shaft 78 that may be considered as being part of a delivery device. The spiral ligation device 74 is releasably secured to the delivery shaft 78 via a first locking feature 80 that is formed as part of the attachment member 76 and a second locking feature 82 that is formed as part of the delivery shaft 78. In some instances, the first locking feature 80 and the second locking feature 82 engage to provide a connection by which the spiral ligation element 74 may be translated and/or rotated by translating and/or rotating the delivery shaft 78 as long as the first locking feature 80 and the second locking feature 82 are held together. In some instances, the LAA ligation device 72 may include a sheath (not shown) that may be moved between an advanced position in which the sheath covers both the first locking feature 80 and the second locking feature 82, thereby holding the first locking feature 80 and the second locking feature 82 together, and a retracted position (as shown) in which the sheath does not cover the first locking feature 80 or the second locking feature 82, and as such the first locking feature 80 may be free to move relative to the second locking feature 82. Accordingly, the spiral ligation element 74 may be released from the delivery shaft 78 simply by retracting the sheath relative to the spiral ligation element 74.

In some instances, the LAA ligation device 74 may include an additional sealing material 84 that helps to seal against possible leaks. The sealing material 84 may be disposed relative to the spiral ligation device 74, for example. In some instances, the sealing material 84 may include a polymeric material such as PET (polyethylene terephthalate). In some instances, the sealing material 84 may include a biopolymer such as collagen. In some instances, the sealing material 84 may include a hydropolymer, a gel or a foam, for example. Other materials are also contemplated. The sealing material 84 may be delivered to the LAA 10 as part of the LAA ligation device 74. In some instances, the sealing material 84 may be delivered via a separate device, either before or after the LAA ligation device 74 is delivered and positioned within the LAA 10.

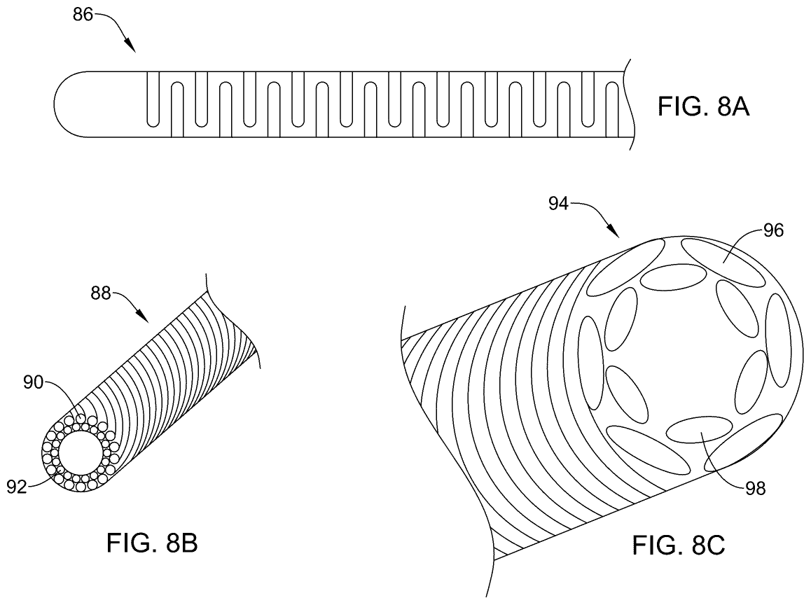

In some instances, the delivery shaft 78 may be adapted to be a flexible shaft with good torque transmission. In some instances, the delivery shaft 78 may be adapted to provide a 1 to 1 relationship, or close to a 1 to 1 relationship, between rotation at a proximal end of the delivery shaft 78 and the corresponding rotation at a distal end of the delivery shaft 78. In some instances, parts of the delivery shaft 78 may include a laser cut hypotube 86, as shown in FIG. 8A. In some instances, parts of the delivery shaft 78 may include a dual wound shaft 88, as shown in FIG. 8B. The dual wound shaft 88 includes an outer coil 90 and an inner coil 92, with one of the outer coil 90 and the inner coil 92 wrapped in a clockwise direction and the other of the inner coil 90 and the outer coil 92 wrapped in a counterclockwise direction. This means that regardless of which way the dual wound shaft 88 is rotated, one coil will expand while the other coil contracts. In some instances, parts of the delivery shaft 78 may include a dual wound shaft 94 as shown in FIG. 8C. The dual wound shaft 94 includes an outer coil 96 and an inner coil 98, with one of the outer coil 96 and the inner coil 98 wrapped in a clockwise direction and the other of the outer coil 96 and the outer coil 98 wrapped in a counterclockwise direction. In some instances, the delivery shaft 78 may be pre-shaped (passively steerable). In some instances, the delivery shaft 78 may be actively steerable.

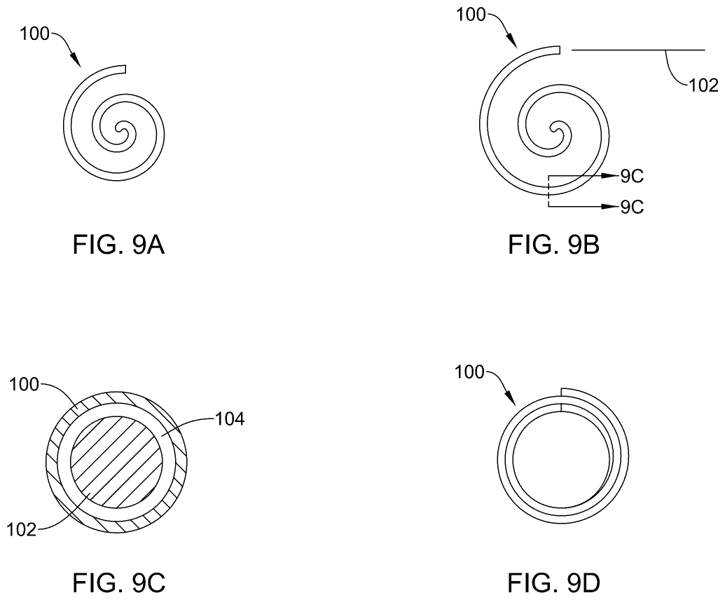

In some instances, an LAA ligation device may include a reshapable spiral ligation element. FIG. 9A shows a spiral ligation element 100 that is biased to a tightly wound configuration. FIG. 9B shows the spiral ligation element 100 expanded to a less tightly wound configuration by virtue of extending a stiff wire 102 within the spiral ligation element 100. As seen in FIG. 9C, which is a cross-sectional view taken along the line 9C-9C of FIG. 9B, the spiral ligation element 100 includes a lumen 104 that accommodates the stiff wire 102. In some instances, the spiral ligation element 100 may be delivered to the LAA 10, extended through the ostium 16 of the LAA 10, and wrapped around the exterior of the LAA 10 with the stiff wire 102 disposed within the lumen 104. Once the LAA 10 has been closed off, the stiff wire 102 may be removed. FIG. 9D shows the spiral ligation element 100 after the LAA 10 has been closed off and the stiff wire 102 has been removed. It can be seen that in FIG. 9D, the spiral ligation element 100 has a more tightly wound configuration than shown in FIG. 9B, but perhaps not as tightly wound as that shown in FIG. 9A. In some instances, inclusion of the LAA 10 cinched down within the spiral ligation element 100 may result in changes to the profile of the spiral ligation element 100 in FIG. 9D relative to that shown in FIG. 9A.

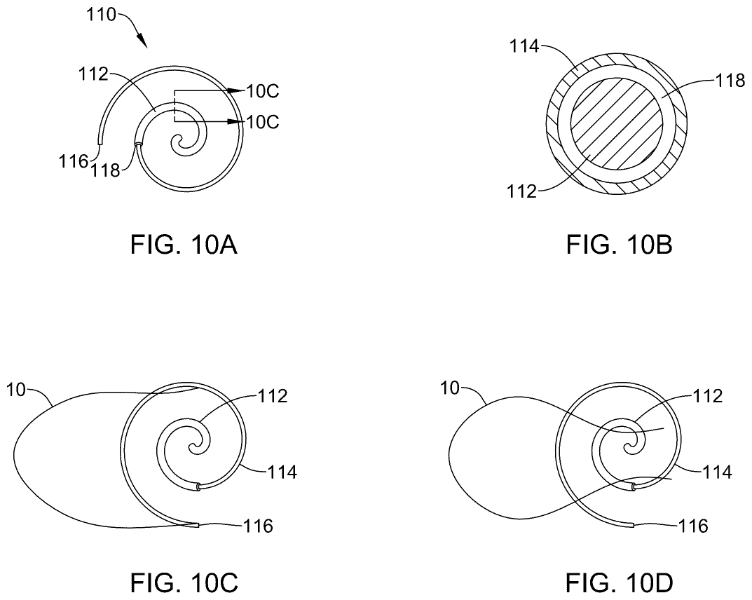

FIGS. 10A through 10D are schematic views of an illustrative two-part LAA ligation device 110. The LAA ligation device 110 may be considered as being adapted for ligating the LAA 10 via a trans-septal approach. In some instances, the LAA ligation device 110 includes a first spiral element 112 and a second spiral element 114. The second spiral element 114 includes an end point 116 that may be adapted for penetrating through the ostium 16 of the LAA 10. In some instances, the first spiral element 112 may be considered as being a permanent ligation spiral, meaning that the first spiral element 112 will remain with the LAA 10 after the LAA ligation device 110 has been deployed and cinched down onto the LAA 10. In some instances, the second spiral element 114 may be considered as being a temporary procedural spiral, meaning that the second spiral element 114 is used during the process of ligating the LAA 10 but is subsequently removed from the LAA 10 (and the patient). As seen in FIG. 10B, which is a cross-sectional view taken along the line 10B-10B of FIG. 10A, the first spiral element 112 includes a lumen 118 that is adapted to accommodate the second spiral element 114 within the first spiral element 112.

In some instances, inclusion of the second spiral element 114 results in an overall larger spiral that may be used to ligate the LAA 10. In some instances, the second spiral element 114, or at least the end point 116 thereof, may be adapted to penetrate the ostium 16 of the LAA 10 and to wind around an exterior of the LAA 10 as the LAA ligation device 110 is rotated. Eventually, as the spiral is wrapped around the LAA 10, the first spiral element 112 will engage and then cinch down on the LAA 10 as the LAA ligation device 110 is further rotated. FIG. 10C shows the LAA ligation device 110 disposed relative to the ostium 16 of the LAA 10 prior to ligating the LAA 10 while FIG. 10D shows the end of the ligation process. It can be seen in FIG. 10C that the second spiral element 114 is just beginning to engage the ostium 16 of the LAA 10 while in FIG. 10D, the first spiral element 112 is now cinched down onto the ostium 16 of the LAA 10. In some instances, the first spiral element 112 may be biased to a first configuration, and the second spiral element 114 may be adapted to cause the first spiral element 112 to expand to a second configuration when the second spiral element 114 extends through the first spiral element 112.

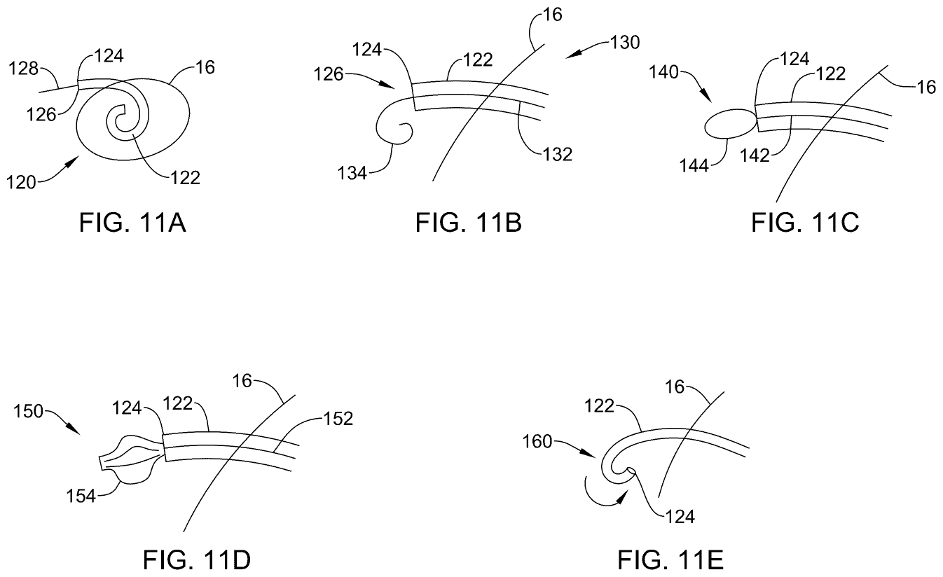

FIG. 11A is a schematic view of an illustrative LAA ligation device 120 shown disposed within the LAA 10. The illustrative ligation device 120 includes a spiral ligation device 122 including an end point 124. In some instances, as shown, the spiral ligation device 122 includes a lumen 126 that is adapted to accommodate a penetration element 128. The penetration element 128 may be disposed within the lumen 126, and may be used to penetrate the ostium 16 of the LAA 10 in order to form an aperture that the end point 124 of the spiral ligation device 122 may extend through in order to reach from an interior of the LAA 10 to an exterior of the LAA 10. In some instances, once the aperture has been formed, the penetration element 128 may be withdrawn. In some instances, the penetration element 128, when present within the lumen 126, may alter an overall configuration of the spiral ligation device 122 (similar to the effect of the stiff wire 102).

In some instances, there may be a desire to provide the end point 124 of the spiral ligation device 122 with an atraumatic tip to reduce the possibility of damaging other tissue while ligating the LAA 10. FIGS. 11B through 11E provide illustrative but non-limiting examples of suitable atraumatic tips. In some instances, the atraumatic tips may be secured to an end of an elongate member. In FIG. 11B, an atraumatic member 130 has been advanced through the lumen 126 in place of the penetration element 128. In some instances, the atraumatic member 130 includes an elongate member 132 and a pigtail element 134 disposed at a distal end of the elongate member 132. The pigtail element 134 may be an integral part of the elongate member 132, or may be separately formed and attached to the elongate member 132.

In FIG. 11C, an atraumatic member 140 has been advanced through the lumen 126 in place of the penetration element 128. In some instances, the atraumatic member 140 includes an elongate member 142 and a balloon 144 disposed at a distal end of the elongate member 142. In some instances, the balloon 144 may be an inflatable balloon, and may be inflated for example using saline or another suitable fluid provided through the elongate member 142. In FIG. 11D, an atraumatic member 150 has been advanced through the lumen 126 in place of the penetration element 128. In some instances, the atraumatic member 150 includes an elongate member 152 and an expandable member 154 disposed at a distal end of the elongate member 152. In some instances, the expandable member 154 may be a nitinol cage or ball that has an expanded “remembered” configuration. In some instances, other shape memory materials such as shape memory polymers may be used in forming the expandable member 154. In FIG. 11E, once the penetration element 128 has been removed, a distal region 160 of the spiral ligation device 122 may be adapted to itself revert to a pigtail shape that provides an atraumatic tip.

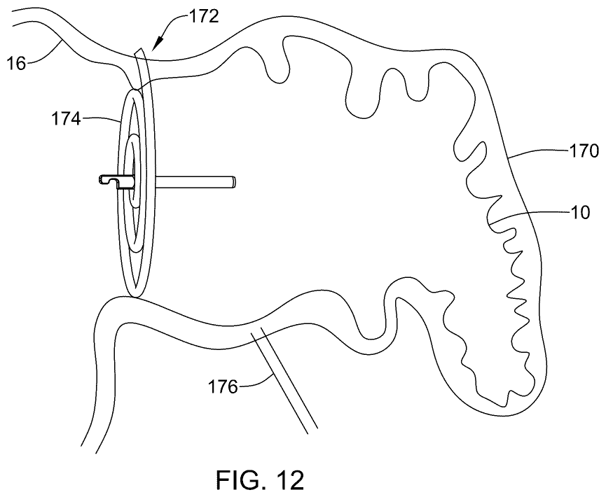

In some instances, it is possible that the process of ligating the LAA 10 using one of the LAA ligation devices described herein may cause blood to leak from inside the LAA 10 to a position outside of the heart. As seen in FIG. 12, in some instances, blood may collect within the pericardium 170 when an end point 172 of a spiral ligation device 174 penetrates the ostium 16 of the LAA 10. In some instances, a precautionary process of accessing an interior of the pericardium 170 with a needle 176 fluidly coupled with an aspiration source outside of the patient may be provided in order to capture any blood that may leak out of the LAA 10 and into the pericardium 170. In some instances, the needle 176 may be advanced between two of the patient's ribs in order to reach an interior of the pericardium 170. In some instances, the needle 176 may be placed under fluoroscopic guidance, for example.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear-elastic” or “non-super-elastic” which, although may be similar in chemistry to conventional shape memory and super elastic varieties, may exhibit distinct and useful mechanical properties. Linear elastic and/or non-super-elastic nitinol may be distinguished from super-elastic nitinol in that the linear elastic and/or non-super-elastic nitinol does not display a substantial “super-elastic plateau” or “flag region” in its stress/strain curve like super-elastic nitinol does. Instead, in the linear-elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super-elastic plateau and/or flag region that may be seen with super-elastic nitinol. Thus, for the purposes of this disclosure linear-elastic and/or non-super-elastic nitinol may also be termed “substantially” linear-elastic and/or non-super-elastic nitinol.

In some cases, linear-elastic and/or non-super-elastic nitinol may also be distinguishable from super-elastic nitinol in that linear-elastic and/or non-super-elastic nitinol may accept up to about 2-5% strain while remaining substantially elastic (e.g., before plastically deforming) whereas super-elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear-elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear-elastic and/or non-super-elastic nickel-titanium alloy. The mechanical bending properties of such material may therefore be generally inert to the effect of temperature over this very broad range of temperature. In some embodiments, the mechanical bending properties of the linear-elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear-elastic and/or non-super-elastic nickel-titanium alloy maintains its linear-elastic and/or non-super-elastic characteristics and/or properties.

In some embodiments, the linear-elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, The devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of the devices described herein in order to define a generally smooth outer surface. In other embodiments, however, such a sheath or covering may be absent. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A method of ligating a left atrial appendage, comprising:
positioning a delivery device to a position within a left atrium proximate the left atrial appendage;
deploying a left atrial appendage ligation device within an ostium of the left atrial appendage, the left atrial appendage ligation device including a spiral ligation element extending from an end point disposed at a periphery of the spiral ligation element to a center point of the spiral ligation element;
rotating the left atrial appendage ligation device relative to the ostium of the left atrial appendage to penetrate through the ostium of the left atrial appendage with the end point of the spiral ligation element to a position exterior to the left atrial appendage; and
after penetrating through the ostium of the left atrial appendage with the end point of the spiral ligation element, rotating the left atrial appendage ligation device relative to the left atrial appendage to rotate the spiral ligation element circumferentially around an exterior of the left atrial appendage.

2. The method of claim 1, further comprising rotating the left atrial appendage ligation device around the exterior of the left atrial appendage to move the left atrial appendage toward the center point of the spiral ligation element, thereby cinching down onto the left atrial appendage.

3. The method of claim 2, further comprising detaching the left atrial appendage ligation device from the delivery device.

4. The method of claim 1, wherein the delivery device is releasably secured to the spiral ligation element.

5. The method of claim 4, wherein the delivery device is releasably secured to the center point of the spiral ligation element.

6. The method of claim 5, wherein the center point of the spiral ligation element includes a first releasable locking feature, and the delivery device includes a second releasable locking feature complementary to the first releasable locking feature.

7. The method of claim 6, wherein the delivery device includes a removable sheath that holds the first releasable locking feature engaged with the second releasable locking feature until the removable sheath is removed.

8. The method of claim 5, wherein the spiral ligation element is held to the delivery device via a breakable connection that is adapted to fail upon application of sufficient torque.

9. The method of claim 1, wherein the delivery device is configured to rotate the left atrial appendage ligation device relative to the left atrial appendage.

10. The method of claim 1, wherein the delivery device comprises a dual-wound coil or a slotted laser-cut hypotube.

11. The method of claim 1, wherein left atrial appendage ligation device includes a polymeric sealing material disposed relative to the spiral ligation element.

12. The method of claim 1, wherein the spiral ligation element is formed from an elongate member having a circular cross-sectional profile.

13. The method of claim 1, wherein the spiral ligation element is formed from an elongate member having a rectilinear cross-sectional profile.

14. A method of ligating a left atrial appendage, comprising:
positioning a delivery device to a position within a left atrium proximate the left atrial appendage;
deploying a left atrial appendage ligation device within an ostium of the left atrial appendage, the left atrial appendage ligation device including:
a spiral ligation element extending from an end point disposed at a periphery of the spiral ligation element to a center point of the spiral ligation element; and
a second spiral element slidingly disposed within a lumen of the spiral ligation element, wherein the second spiral element includes an end point configured to penetrate the ostium of the left atrial appendage;
rotating the left atrial appendage ligation device relative to the ostium of the left atrial appendage to penetrate through the ostium of the left atrial appendage with the end point of the second spiral element to a position exterior to the left atrial appendage; and
after penetrating through the ostium of the left atrial appendage with the end point of the second spiral element, rotating the left atrial appendage ligation device relative to the left atrial appendage to rotate the spiral ligation element circumferentially around an exterior of the left atrial appendage

15. The method of claim 14, further comprising rotating the left atrial appendage ligation device around the exterior of the left atrial appendage to move the left atrial appendage toward the center point of the spiral ligation element, thereby cinching down onto the left atrial appendage.

16. The method of claim 15, further comprising withdrawing the second spiral element from the lumen of the spiral ligation element.

17. The method of claim 16, further comprising detaching the left atrial appendage ligation device from the delivery device.

18. The method of claim 16, wherein the end point of the spiral ligation element is adapted to curl into an atraumatic tip when the second spiral element is withdrawn from the lumen.

19. The method of claim 16, further comprising advancing an atraumatic element through the lumen to extend out of the lumen at the end point of the spiral ligation element subsequent to removal of the second spiral element.

20. The method of claim 14, wherein the spiral ligation element is biased to a first configuration, and the second spiral element is adapted to cause the spiral ligation element to expand to a second configuration when the second spiral element extends through the lumen of the spiral ligation element.
